(19) United States
(12) Patent Application Publication
(10) Pub. No.: US 2006/0293727 A1
(43) Pub. Date: Dec. 28, 2006

(54) System and method for treating exposed tissue with light emitting diodes

(76) Inventors: Greg Spooner, Kensington, CA (US); David A. Gollnick, San Francisco, CA (US); Dean A. MacFarland, Magnolia, MA (US)

Correspondence Address: STALLMAN & POLLOCK LLP, 353 SACRAMENTO STREET, SUITE 2200, SAN FRANCISCO, CA, 94111, US

(21) Appl. No.: 10/422,261

(22) Filed: Apr. 24, 2003

Related U.S. Application Data
(60) Provisional application No. 60/379,350, filed on May 9, 2002.

(51) Int. Cl. A61N 5/06

(52) U.S. Cl. 607/88

(57) ABSTRACT

The invention comprises a system and method for treating an exposed tissue of a patient with a light energy. A plurality of light emitting diodes are disposed over an area of a supporting structure. The light emitting diodes emit light energy. The light energy comprises a substantial band of wavelengths between about 380 and 800 nm. The light emitting diodes are optically coupled to the exposed tissue of the patient. A driver circuit is electrically coupled to the light emitting diodes for driving a current through the plurality of light emitting diodes. An average irradiance of the light energy emitted from the area by the light emitting diodes is at least about 30 mW per square centimeter during a treatment. In some embodiments, the light energy emitted from a first light emitting diode substantially overlaps with light energy emitted from several adjacent light emitting diodes as the light energy propagates toward the tissue. A substantially uniform irradiance profile distribution forms near a surface of the tissue.

### PRIORITY

The present application claims the benefit of U.S. Provisional Application Ser. No. 60/379,350, filed May 9, 2002 titled SYSTEM AND METHOD FOR TREATING EXPOSED TISSUE WITH LIGHT EMITTING DIODES, which is incorporated herein by reference.

### BACKGROUND OF THE INVENTION

Previous techniques for treating tissue with light have included the use of light energy emitted from lasers, flash lamps, and light emitting diodes (LEDs). Lasers can be expensive to manufacture, difficult to build and maintain, and subjected to greater government regulation than other light sources of light energy. The relatively higher cost of lasers results in a higher system cost to the user. Laser systems often benefit from precision alignment of optical components, and this precision alignment increases system cost. Manufacturing yields can be lowered as a result of stringent alignment tolerances often associated with lasers. Optical systems using lasers often call for field service support, and this support can become expensive. In many applications, coherent light energy is not necessary.

Other approaches have relied upon light from sources such as flash lamps and metal halide lamps, instead of lasers. This approach has the advantage of a less expensive, reasonably portable light source, but lamps create their own problems. It is difficult to deliver light from a lamp to the skin. The reflectors that surround lamps and collect the light and direct it to the skin are often precisely built and calibrated. Errors can produce hot spots in the spatial energy distribution. Energy distribution errors can lead to under-treatment in some areas and burning in other areas. Moreover, the spectrum of the light energy from lamps is broad, usually including the visible and stretching into the longer infrared wavelengths. In many instances much of the light energy made with this broad spectrum light source is wasted as many of the wavelengths of light produced are not useful for treatment. In some instances these extraneous light energies can be problematic. The longer wavelengths of light energy are substantially absorbed by water that occupies the skin. Thus, the light from these sources tends to penetrate very poorly, which leads to higher overall light fluence levels to sufficiently treat deeper lying structures. A concomitant risk is burning or damaging the skin. Although optical filters and the like may be used with lamp systems, these optical filters increase the cost and size, and decrease the reliability and efficiency of a lamp based system.

Although light emitting diodes (LEDs) have been used to treat tissue with light, systems using LEDs have typically provided limited amounts of light power and energy delivered to a treatment site. The limited energy emitted by previous LED systems has limited the commercial value of therapeutic treatment with such systems. For example, patients typically do not want treatments lasting over an hour. If an LED based system takes several hours to treat a patient, a prospective patient may elect not to undergo treatment.

Attempts have been made to circumvent problems associated with the low light power levels emitted by LEDs with optical delivery systems. Optical delivery systems can focus light from one or many LEDs to increase the flux density of light energy applied to the tissue. For example, many commercially available LEDs include a curved refracting surface that decreases a divergence of a beam of emitted light energy. It has been proposed that several LEDs having curved refracting surfaces be directed toward a focal point to provide overlapping beams of light. These attempts have not to date been fully successful, so that laser systems are preferred for many therapies despite their high cost and maintenance disadvantages.

The present invention provides a cost effective solution for treating tissue with light emitting diodes that avoids many of the above mentioned problems.

### BRIEF SUMMARY OF THE INVENTION

The invention provides improved systems and methods for treating an exposed tissue with light energy.

In a first aspect the invention comprises a system for treating a tissue of a patient with light. A plurality of light emitting diodes are distributed across an area of a supporting structure. The light emitting diodes emit a light energy. The light energy comprises a central wavelength between about 380 and 800 nm. The light emitting diodes are optically coupled to the tissue of the patient. A driver circuit is electrically coupled to the light emitting diodes for driving the plurality of light emitting diodes. An average irradiance of the light energy emitted from the area by the light emitting diodes is at least about 30 mW per square centimeter during a treatment.

In specific embodiments, the central wavelengths of the light emitting diodes are the same. In many embodiments, the light energy emitted from a first light emitting diode substantially overlaps with light energy emitted from several adjacent light emitting diodes as the light energy propagates toward the tissue. A substantially uniform irradiance distribution profile forms near a surface of the tissue. A dimension across the substantially uniform irradiance distribution profile can be at least about half of the dimension across the area on which the light emitting diodes are disposed, and a dimension across the area can be at least about two centimeters. In a specific embodiment the tissue is a skin tissue and the treatment comprises an acne treatment. The treatment can comprise a cumulative treatment fluence emitted from the area of at least about 50 Joules per square centimeter, and the average irradiance emitted from the area can be at least about 50 mW per square cm during the treatment.

In some embodiments, a driver circuit shifts a central wavelength of the light energy emitted by at least some of the light emitting diodes toward a peak in an intensity of a fluorescence of a proto-porphyrin molecule located within the skin of the patient.

In another aspect, the present invention comprises a method for treating a tissue of a patient with light. A plurality of light emitting diodes are optically coupled to the tissue of the patient. Light energy comprising a central wavelength between about 380 and 800 nm is emitted from a plurality of light emitting diodes distributed across an area of a supporting structure. An average irradiance emitted from the area is at least about 30 mW per square centimeter during a treatment.

In some embodiments, the central wavelengths of the light emitting diodes may be the same. The light energy emitted from a first light emitting diode may substantially overlap the light energy emitted from several adjacent light emitting diodes. As the light energy propagates toward the tissue a substantially uniform energy distribution profile of irradiation may form near a surface of the tissue. A dimension across a tissue treatment area may be at least about half of a dimension across the area where the light emitting diodes are distributed, and a dimension across the area where the light emitting diodes are distributed may be at least about two centimeters. In specific embodiments the tissue is a skin tissue and the treatment comprises an acne treatment. In some embodiments, the treatment may comprise a cumulative treatment fluence emitted from the LED area of at least about 50 J/cm², and the average irradiance may be at least about 50 mW/cm² at a tissue surface during the treatment.

In specific embodiments a shifting of a central wavelength of the light energy emitted by at least some of the light emitting diodes is toward a peak in an intensity of a fluorescence of a proto-porphyrin molecule located in the skin of the patient.

### BRIEF DESCRIPTION OF THE DRAWINGS

FIG. 1 illustrates a system including a console and remote head emitting light energy for treating a tissue of a patient.

FIG. 2 illustrates an enlarged view of several LEDs on a surface of the remote head.

FIG. 3 illustrates a pattern of light energy from an LED as the light energy travels toward a tissue structure.

FIG. 4A illustrates light penetration depth in a tissue for different wavelengths of light energy.

FIG. 4B illustrates a fluorescence spectra of a molecular structure at different depths in a tissue.

FIG. 5 illustrates a preferred embodiment of a remote head for delivery of light energy to an exposed tissue.

FIG. 6. illustrates a schematic diagram of a system for treating tissue with a light energy emitted by LEDs.

FIG. 7 illustrates a user interface of a system as in FIGS. 1 and 6.

FIG. 8 illustrates a preferred embodiment of a printed circuit board having many ballast circuits and LEDs mounted thereon for emitting a light energy.

FIG. 8A schematically illustrates a ballast circuit.

FIG. 8B schematically illustrates an exemplary embodiment of a printed circuit board formed as a paddle for treating a cheek of a patient.

FIG. 9 illustrates several LEDs emitting a spatially overlapping light energy.

FIG. 10 illustrates several energy irradiance distribution profiles for spatially overlapping beams of light energy as illustrated in FIG. 9.

FIGS. 11A-11C illustrate cumulative irradiance distribution profiles at a tissue surface in accord with an aspect of the present invention.

FIG. 12 illustrates a cross sectional view of a printed circuit board as illustrated in FIG. 8 mounted to a water cooled heat sink.

FIGS. 13A-13E illustrate the geometrical layouts of several LED ballast circuits.

FIGS. 14A and 14B illustrate an array of geometric lenses for collimating light energy emitted from LEDs mounted on a circuit board.

FIGS. 15A and 15B illustrate an array of diffractive lenses for collimating light energy emitted from LEDs mounted on a circuit board.

FIG. 16 illustrates an emission of optical power from an LED as a function of current passing through the LED.

FIGS. 16A-16E illustrate irradiance profile distributions for irradiance at a tissue surface in accord with the present invention.

FIGS. 17A and 17B illustrate a change in a central wavelength of light energy emitted from an LED as a function of current passing through the LED.

FIG. 18 illustrates an emission of optical power from an LED as a function of current passing though the LED for an LED changing a peak wavelength of emission as in either of FIGS. 17A and 17B.

FIGS. 19A-C illustrate pulsed currents for increasing peak optical power emitted by LEDs to shift a central wavelength of light energy emitted from an LED.

### DETAILED DESCRIPTION OF THE INVENTION

The present invention provides improved systems and methods for treatment of tissue with light emitted from LEDs. By closely positioning several (often several hundred) LEDs, therapeutic levels of light energy can be achieved with LEDs without using expensive and complex light delivery systems. By achieving high energy densities without a complex delivery system, system cost is kept to desirably low levels to advantageously permit commercial use of LED treatment systems. The present invention makes use of desirable LED properties such as use of selected wavelengths while avoiding the disadvantages associated with lasers and lamps.

In an embodiment, the invention includes an apparatus using high flux LED sources to treat acne, acne vulgaris and other forms of active acne. The invention includes a selection of treatment wavelengths based on an understanding of a photodestructively targeted acne bacterium, Propionibacterium Acnes. The invention may also be used for other dermatologic conditions requiring and benefiting from non-thermal phototherapy at specific ultraviolet (UV) to near infrared (IR) wavelengths of non-coherent light. Examples include the treatment of hyperbilirubinemia, general photodynamic therapy with or without exogenous photosensitizers. The apparatus is applicable in dermatologic and other directly accessible tissues, for example any exposed tissue.

The output light energy from an LED typically comprises light energy having several wavelengths near a central emission wavelength. The spectrum of wavelengths of a light energy emitted from a light emitting diode is often characterized as having a full width half maximum (FWHM) value based on the wavelengths at which the output energy intensity is half of a peak output intensity. A difference in wavelength between the two wavelengths having half of the peak output energy intensity is referred to as the full width half maximum (FWHM). Typical values of the full width half maximum of an emission spectrum for an LED range from about 5 nm to 20 nm. A central wavelength of an emitted light energy having several wavelengths encompasses a wavelength of a centroid of an emission spectrum of the emitted light energy. A substantial band of wavelengths of a light energy emitted from an LED encompasses a range of wavelengths included in the full width half maximum of an emission spectrum of an LED.

As used herein a substantially uniform irradiance distribution profile encompasses an irradiance distribution that remains within about 25% of a peak irradiance. A substantially constant irradiance encompasses an irradiance that remains within about 25% of a nominal value.

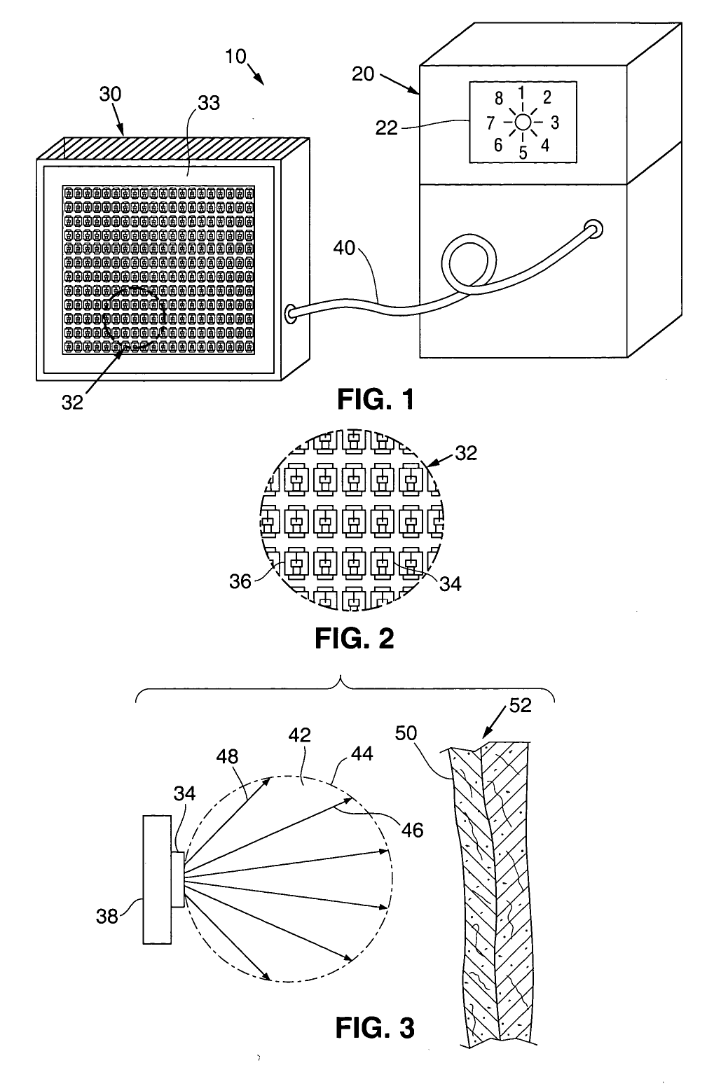

A system 10 for treating tissue with light according to the invention is illustrated in FIG. 1. A console 20 is attached to a remote head 30 by an umbilical cable 40. Cable 40 operationally connects remote head 30 with console 20 of system 10. The remote head 30 includes several LEDs 32 for emitting light energy. An enlarged view of a region comprising several LEDs 32 of remote head 30 is illustrated in FIG. 2. Individual LEDs 34 and 36 are shown in FIG. 2.

Console 20 includes a user interface 22 for controlling a treatment. The console 20 preferably includes circuits for controlling the treatment. Circuits for controlling the treatment include LED driver circuits for controlling a current passing through the LEDs, and a feedback circuit for verifying that the LEDs are operational. The console 20 preferably includes apparatus for cooling remote head 30.

As illustrated in FIG. 3, an individual LED 34 is included within a body 38. The LED 34 emits beam of a treatment light energy 42 for treating a tissue 52 having an exposed surface 50. The beam 44 of treatment light energy 42 diverges as it approaches tissue 52. The divergence of beam 44 is illustrated by rays 46 and 48.

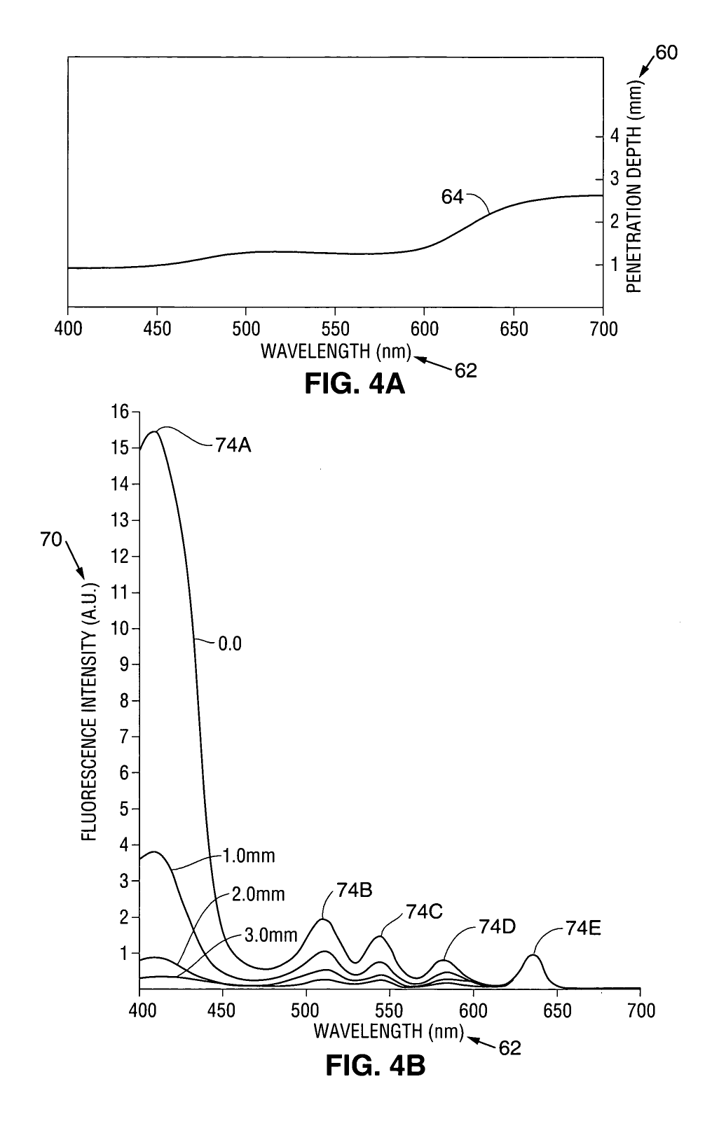

A penetration depth 60 of a treatment light energy 42 into a tissue is illustrated in FIG. 4A. A depth of penetration 60 of light energy into a tissue varies with a wavelength 62 of the light energy. As shown in FIG. 4A, light energy having a shorter wavelength penetrates less deeply into tissue than light having a longer wavelength. A plotted curve 64 of light energy penetration depth for several wavelengths of light energy is shown in FIG. 4A. For example, light having a wavelength of 400 nm penetrates to a depth of about ½ mm and light having a wavelength of about 650 nm penetrates to a depth of about 2 mm. Although FIG. 4A illustrates a general trend, actual values of light energy penetration depths for a particular wavelength vary significantly among individual patients. For example, patients having a darker skin color will typically show a decreased penetration depth for shorter wavelengths of light energy.

During treatment of a tissue 52, a fluorescence of molecules within tissue 52 typically occurs. An amount of tissue fluorescence occurring during a treatment will typically vary with both a wavelength of a light energy applied to the tissue and a depth into the tissue. In specific embodiments, a specific fluorescent molecule may be targeted, for example proto-porphyrin IX (PP-IX). Direct phototherapy is useful in treating acne vulgaris. This therapy typically includes the photoactivation of proto-porphyrin IX (PP-IX), a common metabolic product. PP-IX is found in relatively large amounts in living Propionibacterium Acnes, the bacterium principally responsible for acne vulgaris.

The fluorescence spectrum of proto-porphyrin IX has been well characterized and can be combined with properties of light penetration into a tissue as in FIG. 4A to estimate an effectiveness of a treatment at a particular wavelength. Referring to FIG. 4B, an intensity of fluorescence 70 at a wavelength 62 of light energy 42 is illustrated at tissue depths of 0.0, 1.0, 2.0 and 3.0 mm for proto-porphyrin IX. An intensity of fluorescence 70 changes with wavelength 62 of light energy. Several peaks 74A-74E of an intensity of fluorescence 70 are illustrated. A wavelength at which a peak occurs remains relatively constant for each tissue depth shown. For example, the peak 74A occurs at about 410 nm at penetration depths of 0.0, 1.0, 2.0 and 3.0 mm. As shown in FIG. 4B, the fluorescence intensity peak 74A at about 410 nm has the greatest change in intensity at varying depths within a tissue. Alternatively, an intensity of a fluorescence peak 74E occurring at about 635 nm changes very little with wavelength 62 of applied light energy. As with penetration depth, FIG. 4B illustrates a general trend and a fluorescence intensity for a particular wavelength and an individual patient can vary significantly among patients.

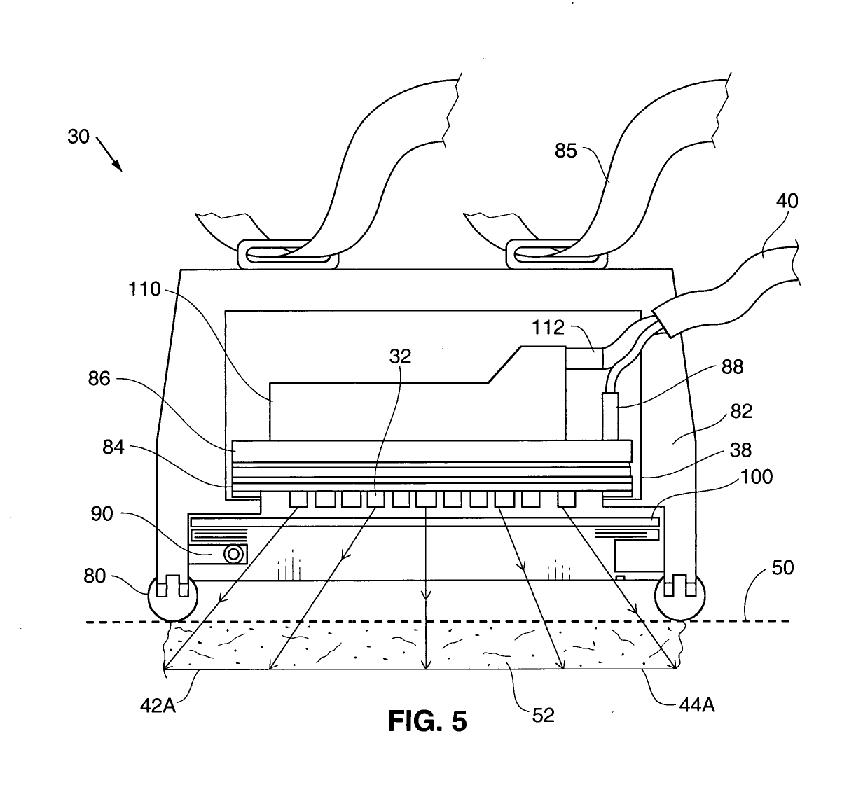

A preferred embodiment of a remote head for applying a light energy to a patient is illustrated in FIG. 5. This embodiment is preferably used to treat flat areas of skin, such as a forehead, cheeks and a back of a patient. Alternate embodiments provide appropriate shapes and designs for treating curved areas of skin such as a facial mask for treating a face of a patient. The remote head 30 includes a body 38 comprising several LEDs 32 for emitting a beam 44A of a treatment light energy 42A. A treatment light beam 42A comprises several individual spatially overlapping light beams such as individual light beam 42 emitted by an individual LED 34 as described above. The beam 44A is directed to a surface 50 of a treated tissue 52. An elastomer foam material 80 formed into an annular ring for contacting an exposed tissue surface 50 supports the head 30 against tissue surface 50 and maintains a fixed distance between LEDs 32 and tissue surface 50. The LEDs 32 are mounted on a printed circuit board 84. Body 38 preferably comprises metal-clad printed circuit board 84 comprising an aluminum backing 86 for rigidly supporting printed circuit board 84 and conducting heat away from LEDs 32. Alternatively, circuit board 84 may be made flexible. A housing 82 supports the metal-clad printed circuit board 84 and elastomer foam material 80. A connector 88 electronically couples printed circuit board 84 with console 20 via umbilical cable 40. A photo sensor 90 measures an intensity of a light energy 42A emitted by body 38. Photo sensor 90 is operationally coupled to console 20 by cable 40.

A plastic Fresnel lens 100 is positioned between light emitting diodes 32 and tissue surface 50. Plastic Fresnel lens 100 electrically insulates tissue 52 from printed circuit board 84. Plastic Fresnel lens 100 transmits treatment light energy 42A and slightly decreases divergence light beam 44A. Suitable Fresnel lenses are available from many suppliers. For example, Fresnel lens model NT 32-683, available from EDMUND INDUSTRIAL OPTICS of Barrington, N.J., provides transmission of over 90% and has a focal length of about 3 inches.

A water cooled channeled aluminum block 110 conducts heat from and cools metal-clad printed circuit board 84 via umbilical cable 40. Water passing through channels of aluminum block 110 actively cools aluminum block 110. Alternate embodiments may include a thermo-electric cooler for active cooling, and fans for active cooling, and pumps for active cooling. Active cooling systems are well known and additional details of active cooling systems have not been shown to avoid prolixity. Umbilical cable 40 comprises at least one channel for forcing water into aluminum block 110 and at least on channel for removing water from aluminum block 110. Umbilical cable 40 attaches to aluminum block 110 at plumbing connector 112. A Velcro strap 84 attaches remote head 30 to a patient having tissue 52. For example, Velcro strap 84 may be wrapped around an arm of a patient. In various embodiments, as many Velcro straps 84 as needed to attach remote head 30 to a patient may be provided.

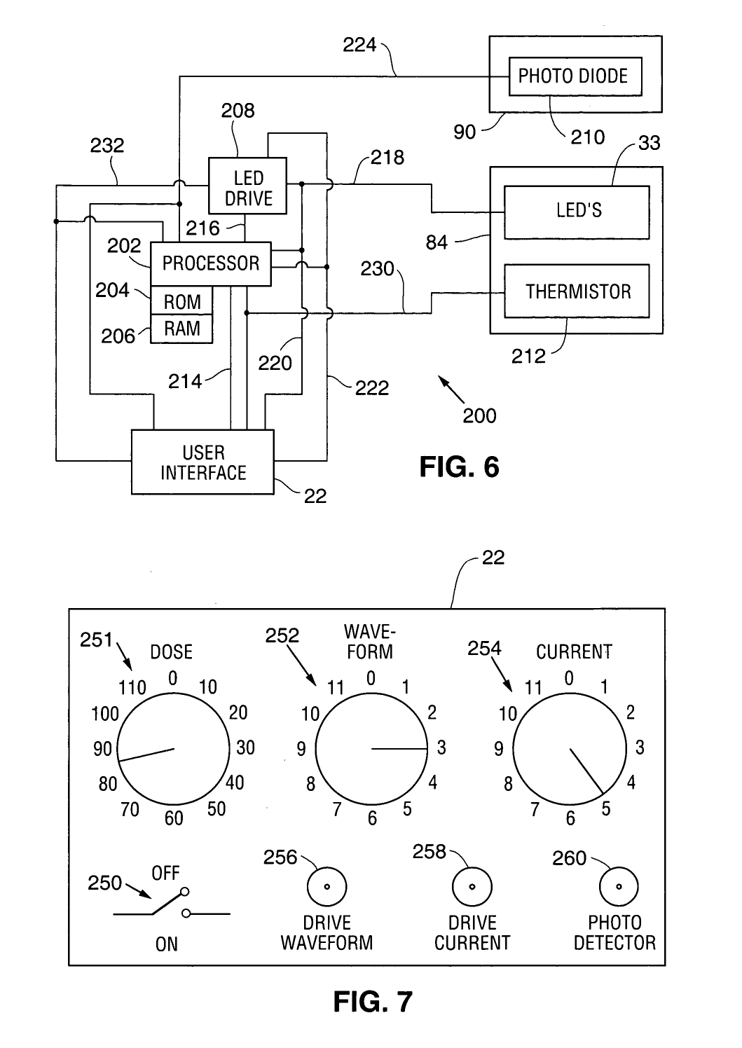

A schematic diagram of an electrical circuit 200 of a preferred embodiment is illustrated in FIG. 6. A processor 202 comprises a random access memory (RAM) 206 and a read only memory (ROM) 204. Other tangible media in addition to RAM 206 and ROM 204 including floppy disk drives and hard disk drives may be included in processor 202. Processor 202 is preferably a PIC 16C76 embedded processor available from Microchip, Inc. of Chandler Ariz. User interface 22 is electronically coupled to processor 202 via electronic communication line 214. An array 33 of LEDs are bonded to metal-clad circuit board 84. A temperature sensor is bonded to metal-clad circuit board 84. The temperature sensor preferably comprises a thermistor 212. In alternate embodiments a temperature sensor may comprise a thermocouple and any transducer converting thermal energy into an electrical signal. Thermistor 212 is electronically coupled to processor 202 and user interface 22 by temperature sensor line 230. In alternate embodiments, thermistor 212 and processor 202 are electrically coupled to an active cooler as described above for controlling a temperature of the semiconductor junctions of LEDs of array 33 at a pre-selected level. An LED drive circuit 208 provides a voltage and current for driving an array 33 of LEDs. An LED drive circuit preferably comprises a constant current power supply capable of driving about 100 chains of ballasted LEDs. A suitable power supply is a modified CW diode driver supply, LDD-1500-30-40, manufactured by Lumina Power, Inc, of Danvers, Mass. LED drive circuit 208 is electronically coupled to processor 202 by LED control line 216. An array 33 of LEDs is electronically coupled to LED drive circuit 208 by LED drive line 218. User interface 22 and processor 202 are electronically coupled to a photo detector 90 comprising a photo diode 210 photo sensor line 224 couples photo detector 90 to processor 202 and user interface 22. In preferred embodiments photo detector 90 includes electrical circuits for amplifying an electrical signal from photo diode 210. In alternate embodiments a photo sensor may comprise a photo transistor and any transducer converting light energy emitted by an LED into an electrical signal. An LED current control line 232 electronically couples user interface 22, processor 202 and LED drive 208. An LED drive current sampling line 222 electronically couples user interface 22, processor 202 and LED drive circuit 208. An LED drive voltage line 220 electronically couples LED drive circuit 208, LED array 33, processor 202 and user interface 22. In an alternate embodiment, an output light irradiance may estimated based on a measured temperature as described above and a drive current passing through the LED array 33. In this embodiment the emitted light energy may calibrated by measuring emitted light energy for various combinations of measured temperature and current waveforms. Processor 202 may comprise an algorithm for estimating an output light irradiance based on a measured temperature and drive current passing through the LED array 33.

An embodiment of user interface 22 is illustrated in FIG. 7. A switch 250 turns on system 10. A dose select knob 251 selects a dose of energy applied to a tissue per unit area of tissue in J/cm². A waveform select knob 252 selects a waveform for driving the LED array 33. Example waveforms selected by knob 252 included continuous wave (CW) and pulsed waveforms. A current select knob 254 controls a current passing through LED array 33. The processor 202 determines an intended total elapsed treatment time to achieve a desired dose as selected by knob 251 based on the waveform selected by knob 252 and the current selected by knob 254. A timer of processor 202 times the treatment, and processor 202 shuts off the current to the LED array 33 in response to the measured treatment time reaching the intended treatment time. User interface 22 includes BNC attachments for measuring electrical signals. For example, Drive Waveform output 256 provides a voltage across LED array 33. Drive Current output 258 provides a voltage related to the current passing through LED array 33. Photo Detector output 260 provides a voltage related to the light energy irradiating photo sensor 90. Alternate embodiments may include using processor 202 to automatically measure a signal of any line attached to processor 202 as described above, automatically interrupting a treatment in response to a measured signal such as an elevated temperature, and may further include a display for showing measured signals.

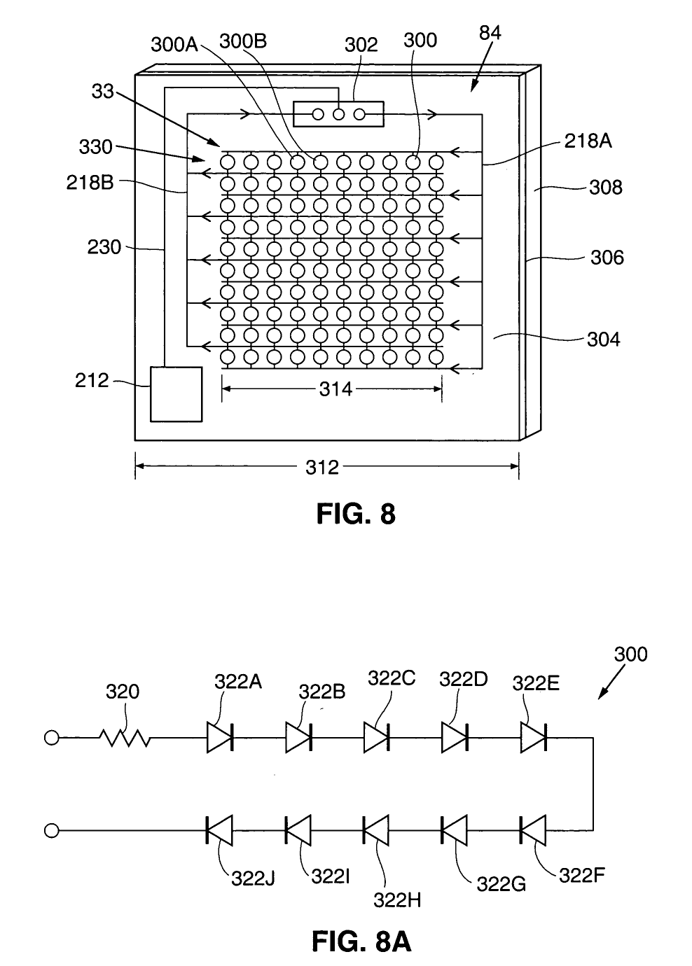

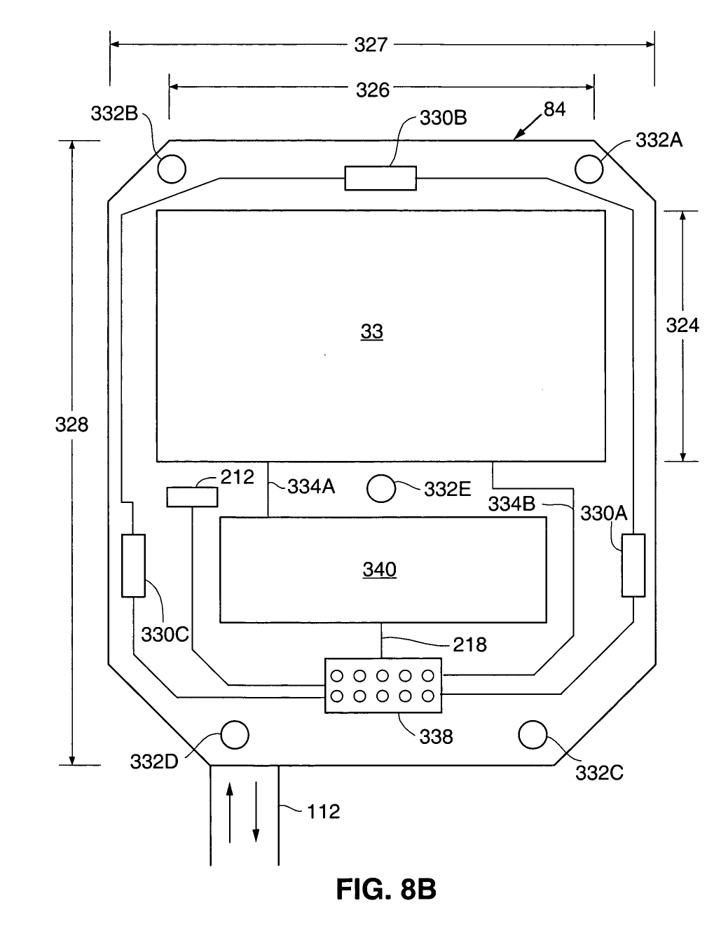

A preferred embodiment of a metal-clad printed circuit board 84 is illustrated in FIG. 8. Metal-clad printed circuit boards are available from many suppliers including the BERGQUIST COMPANY of Chanhassen, Minn. An array 33 of LEDs is mounted on metal-clad printed circuit board 84. A dimension 312 across printed circuit board 84 is preferably about 4 inches. A dimension 314 across array 33 of LEDs is preferably about 3 inches. A temperature sensor comprising a thermistor 212 is mounted on printed circuit board 84. Temperature sensor line 230 is electronically coupled to thermistor 212. A connector 302 attaches to umbilical cable 40 and electronically couples printed circuit board 84 with console 20.

LED drive line 218 comprises a current feed line 218A and a current return line 218B for passing an electrical current in the direction illustrated by arrows shown in FIG. 8. The lines 218A, 218B and 230 are formed in an electrical conducting layer 304 of printed circuit board 84. Printed circuit board 84 comprises dielectric layer 306 formed from a dielectric material for electronically insulating circuits of board 84, and a conducting layer 308 formed in a heat conducting material for conducting heat from board 84. Aluminum is a preferred heat conducting material. A ballast circuit 300 is illustrated in FIG. 8. Ballast circuit 300 is repeated over the array 33 of LEDs, for example ballast circuits 300A and 300B.

A ballast circuit 300 is schematically illustrated in FIG. 8A. A ballast resistor 320 is connected in series with several serially connected LEDs 322A-322J. The number of LEDs connected in series with ballast resistor 320 has a range from about 3 LEDs to 40 LEDs is preferably from about 5 LEDs to 20 LEDs and ideally from about 6 LEDs to 12 LEDs. In alternate embodiments at least one transistor is used in ballast circuit 300 for controlling a current passing serially connected LEDs of a ballast circuit 300.

Returning to FIG. 8, an array of LEDs 33 comprises an array of ballast circuits 330. As illustrated in FIG. 8 an array of ballast circuits comprises 100 ballast circuits connected in parallel. The ballast circuits serve to distribute current evenly between each string of LEDs. Preferably ballast circuit 300 is repeated throughout the array 330 of ballast circuits, including ballast circuits 300A and 300B. A number of ballast circuits on a printed circuit board ranges from about 10 ballast circuits to about 1000 ballast circuits, preferably from about 20 ballast circuits to 500 ballast circuits, more preferably from about 50 ballast circuits to 200 ballast circuits and ideally from about 75 ballast circuits to 150 ballast circuits.

Although preferred values for dimensions 312 and 314 across printed circuit board 84 and array 33 of LEDs, respectively, have been described above, several values are possible. A range of dimension 312 across printed circuit board 84 is from about 1 inch to 20 inches, is preferably from about 2 inches to 10 inches, and ideally from about 3 inches to 5 inches. A range of dimension 314 across array 33 of LEDs is from about ¾ inch to 16 inches, is preferably from about 1.5 inches to 8 inches, and ideally from about 2 to 6 inches.

An exemplary embodiment of a printed circuit board 84 having an array 33 of LEDs for treating acne on a cheek of a patient is illustrated in FIG. 8B. Circuit board 84 is formed in a shape of a paddle. A first dimension 328 across circuit board 84 is about 6 inches. A second dimension 327 across circuit board 84 is about 5 inches. A first dimension 324 across array 33 of LEDs is about 3 inches. A second dimension 326 across array 33 of LEDs is about 4 inches. Several holes 332A-332D are formed in circuit board 84 for mounting board 84 to a housing 82 as described above. An electrical connector 338 is attached to board 84 for reversibly connecting board 84 to umbilical cable 40 as described above. A plumbing connector 112 is attached to an aluminum block mounted on a back surface of board 84 as described above.

One hundred resistors 340 are positioned on board 84. Each of one hundred resistors 340 is electrically connected in series to ten LEDs to form a ballast circuit as described above. Array 33 of LEDs has 1000 LEDs of 100 ballast circuits mounted thereon. Current passes from one hundred resistors 340 to array 33 of LEDs over one hundred current feed lines 334A. One hundred current feed lines 334A electrically couples each of one hundred resistors 340 with 10 LEDs of array 33. Current return line 334B returns current from array 33 of LEDs to connector 338.

Switches 330A, 330B and 330C are mounted on board 84 and electrically coupled to connector 338. Switches 330A, 330B and 330C are closed when the skin to be treated contacts the housing 82 positioned over board 84 and board 84 is mounted in housing 82. Connector 338 and LED drive line 218 electrically couple one hundred resistors 340 to LED drive 208 described above.

Several beams 44A-44G of light energy are emitted by several LEDs 350A-350G as illustrated in FIG. 9. LEDs 350A-350G are mounted on a printed circuit board 84 as described above. LEDs 350A-350G are separated from a tissue surface 50 by a controlled separation distance 354. Beams 44A-44G of light energy are arranged to overlap at a tissue surface 50. The overlap of light beams 44A-44G is preferably arranged to provide a desired irradiance distribution profile at a tissue treatment surface. A point of tissue surface 50A is irradiated by beams 44C and 44D as illustrated in FIG. 9. In practice, several beams irradiate a point 50A on a surface 50 of a tissue 52. For example, an array of LEDs is a two dimensional array, and only one row of an array is illustrated in FIG. 9. By controlling a separation distance between LEDs and a separation distance 354, a desired amount of light beam overlap and a desired irradiance distribution profile are obtained on a tissue surface 50. A broad range of average power intensities can be achieved by appropriately selecting a dimension 314 across LED array 33, a separation distance 354 from a tissue surface to a light source, a number of ballast circuits, a number and type of LEDs within each ballast circuit, a current and a voltage across the ballast circuits and cooling of heat sink. In an embodiment separation distance 354 is about 2.5 cm. A range of values of separation distance 354 is from about 0.2 cm to 20 cm, preferably from about 0.3 to 10 cm, more preferably from about 0.5 to 5 cm and ideally from about 1 to 4 cm.

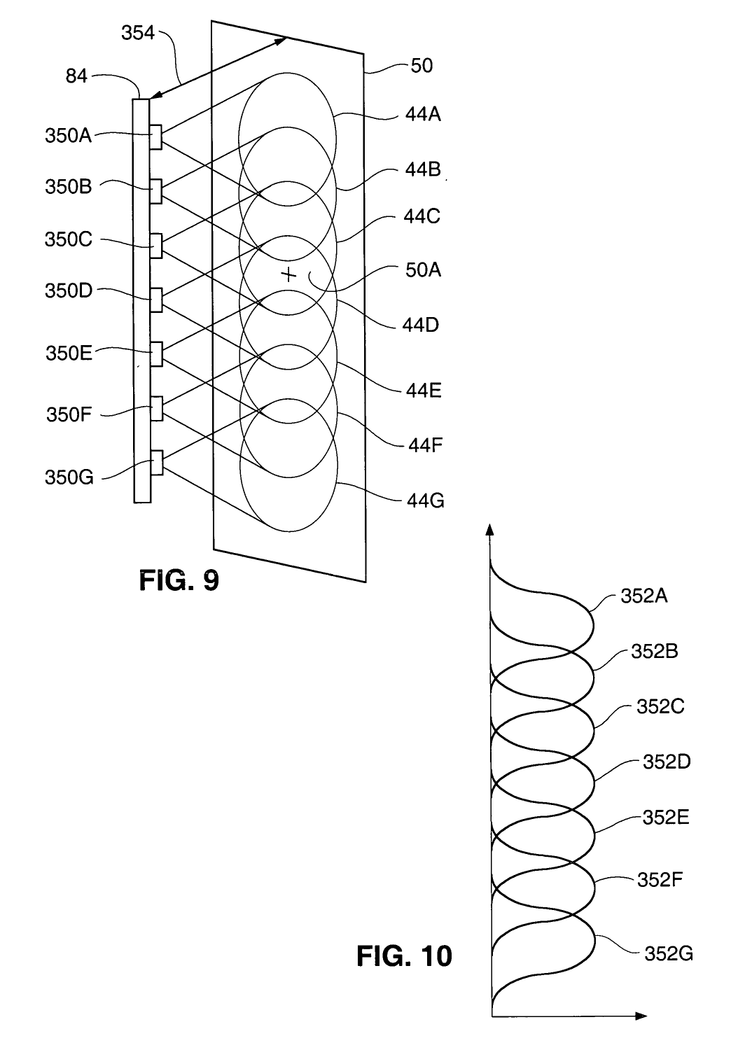

Irradiance distribution profiles for several spatially overlapping beams of energy are illustrated in FIG. 10. Irradiance distribution profiles 352A-352G of beams 44A-44G respectively are illustrated. A cumulative irradiance applied to a point on a surface of a tissue is calculated by adding the irradiance from each beam applied to the point on the tissue surface. The amount of energy applied from a beam to a point on a tissue surface is determined by an intensity from the intensity distribution profile for the beam at the point on the tissue surface. The total amount of energy applied to a point on the tissue surface is the sum of the energy applied to the point by each of the individual beams.

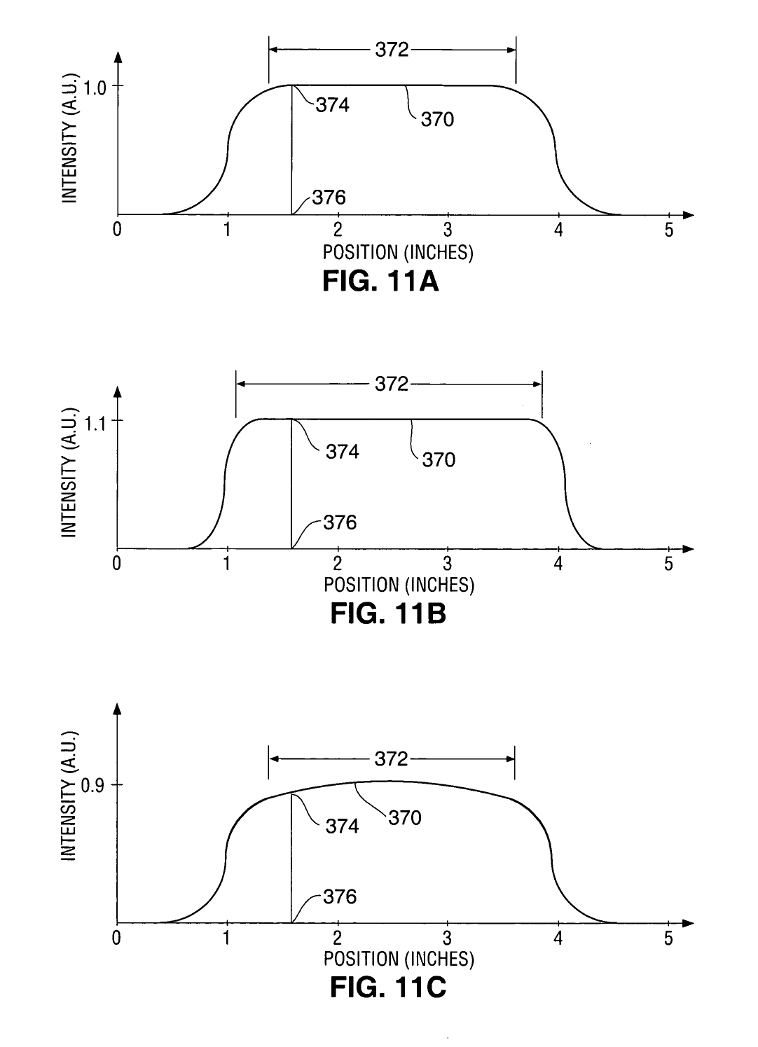

The positions of LEDs on printed circuit board 84 and separation distance 354 are arranged to provide a substantially uniform irradiance profile distribution profile on a tissue surface. For example, FIG. 11A illustrates a uniform irradiance profile distribution 370. A dimension 372 across the uniform irradiance profile distribution 370 is related to a dimension 314 across LED array 33, separation distance 354 and the divergence of beams 44. For an LED array 33 having a dimension 314 across the array of about 3 inches, a dimension 372 across uniform intensity profile distribution is preferably about 2.5 inches. A position 376 on a surface 50 of a tissue 52 is exposed to an intensity 374 of light beam energy. The value of intensity 374 is in arbitrary units (AU) and illustratively 1. A number of LEDs illuminating point 376 on surface 50 of tissue 52 is at least 8, preferably at least 16, more preferably at least 32, even more preferably at least 64 and ideally at least 128.

A uniform irradiance profile distribution 370 on a tissue surface 50 obtained with a separation distance 354 decreased by several millimeters from that of FIG. 11A is illustrated in FIG. 11B. A dimension 372 across uniform irradiance profile distribution 370 has increased. A position 376 on a surface 52 of tissue 50 has an intensity 374 that remains substantially constant and has a value near 1. A number of LEDs illuminating point 376 on surface 50 of tissue 52 at a decreased separation distance 354 is at least 4, preferably at least 8, more preferably at least 16, even more preferably at least 32 and ideally at least 64.

A uniform intensity distribution 370 on a tissue surface 50 obtained with a separation distance 354 increased by several millimeters from that of FIG. 11A is illustrated in FIG. 11C. A dimension 372 across uniform irradiance profile distribution 370 has decreased. A position 376 on a surface 52 of tissue 50 has an intensity 374 that remains substantially constant and has a value of 0.9 and near 1.

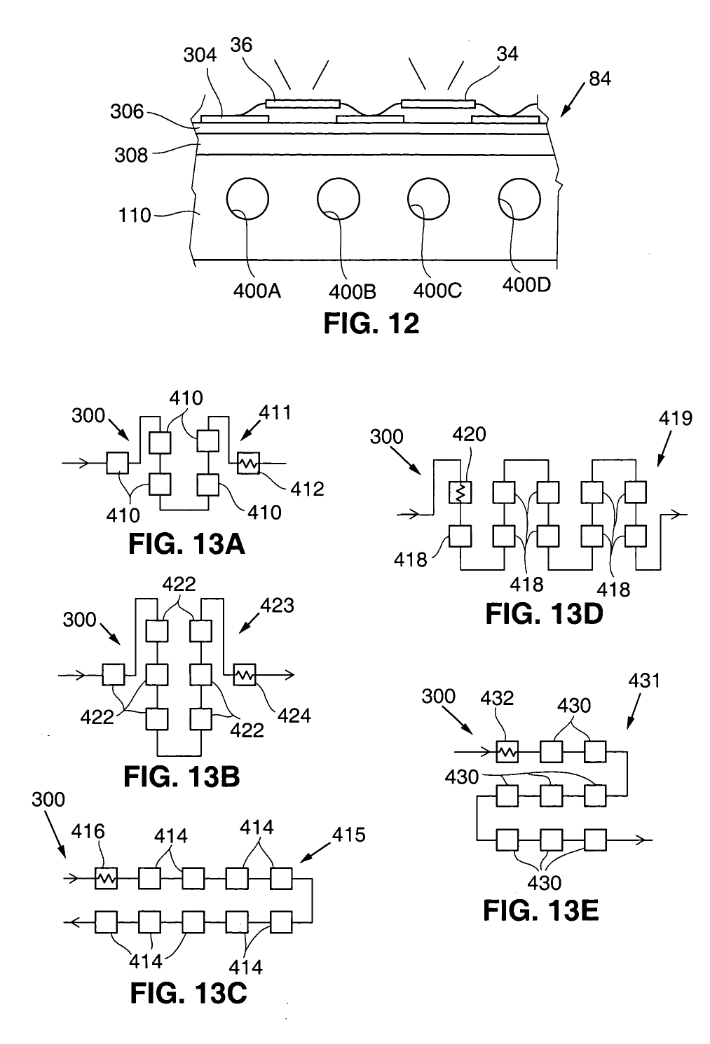

As illustrated in FIG. 12, metal-clad printed circuit board 84 comprises electrical conducting layer 304, dielectric layer 306 and heat conducting layer 308 as described above. several LEDs including LEDs 34 and 36 are electrically connected to conducting layer 304. An aluminum block 110 is thermally bonded to heat conducting layer 308. Channels 400A-400D are formed in aluminum block 110. Channels 400A-400D are filled with a liquid coolant for removing heat from block 110. Channels 400A-400D connect with umbilical cable 40 for removing heated liquid from block 110 of remote head 30 of system 10.

In alternate embodiments of the invention, aggressive cooling techniques may be employed to cool heat generated by electrical components on circuit board 84. For example fluid circulating through channels 400A-400D may have a temperature below the freezing point of water and comprise ethylene glycol. Further, liquid nitrogen may be circulated through channels 400A-400D. Techniques for chilling fluids below the freezing point of water as described above are well known and not described in further detail to avoid prolixity.

Several embodiments of a ballast circuit 300 are illustrated in FIGS. 13A-13E. A ballast circuit comprises several of an LED 410 in series with a ballast resistor 412. Electronic components of ballast circuit 300 are arranged in a geometric pattern 411. Geometric pattern 411 is repeated on the remaining ballast circuits of board 84. Similarly FIGS. 13B-13E illustrate several of LEDs 422, 414, 418 and 430 respectively, in series with a ballast resistors 424, 416, 420 and 432 respectively. In several embodiments of ballast circuit 300, patterns 423, 415, 419 and 431 of FIGS. 13B-13E, respectively are repeated across board 84.

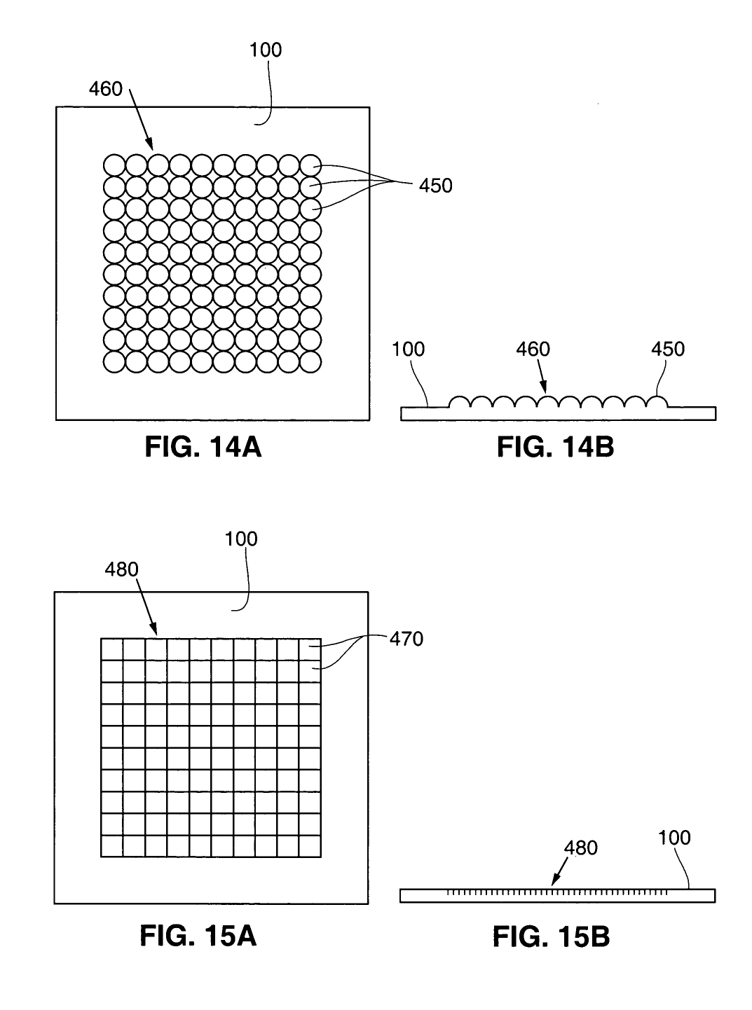

FIGS. 14A and 14B illustrate front and side views of a cover 100 used in an alternate embodiment using a micro array 460 of convex lenses. Several of a convex lens 450 are formed on a surface of cover 100 to form convex lens array 460. Lens 450 decreases a divergence of light beams emitted from light emitting diodes as light energy travels toward tissue surface 50 as described above. Preferably, each lens 450 is registered with each LED. Alternatively, each lens is registered with pattern of each ballast circuit. In an embodiment, convex lens 450 comprises a spherical ball lens, and micro array 460 comprises an array of spherical ball lenses for collimating light energy traveling toward tissue surface 50.

FIGS. 15A and 15B illustrate front and side views of a cover 100 used in an alternate embodiment using a micro array 480 of Fresnel lenses. Several of a Fresnel lens 470 are formed on a surface of cover 100 to form Fresnel lens array 480. Fresnel lens 470 decreases a divergence of light beams emitted from light emitting diodes as light energy travels toward tissue surface 50 as described above. In an alternate embodiment, a micro array 480 of lenses comprises an array of diffractive optical lenses formed in a surface of cover 100. Preferably, each lens 470 is registered with each LED. Alternatively, each lens is registered with a pattern of each ballast circuit as described above.

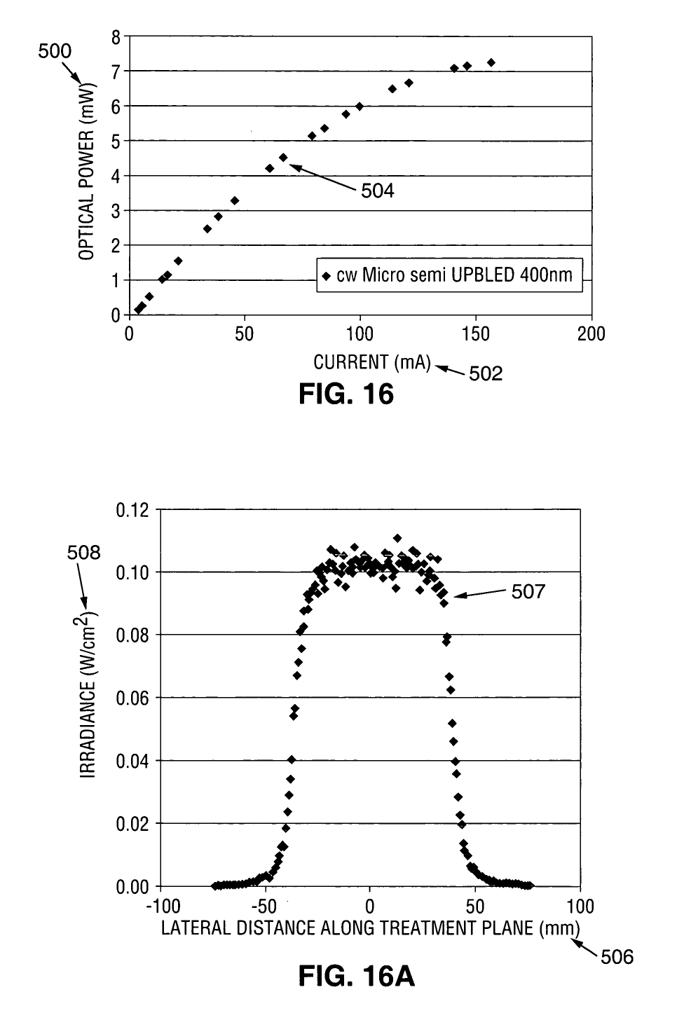

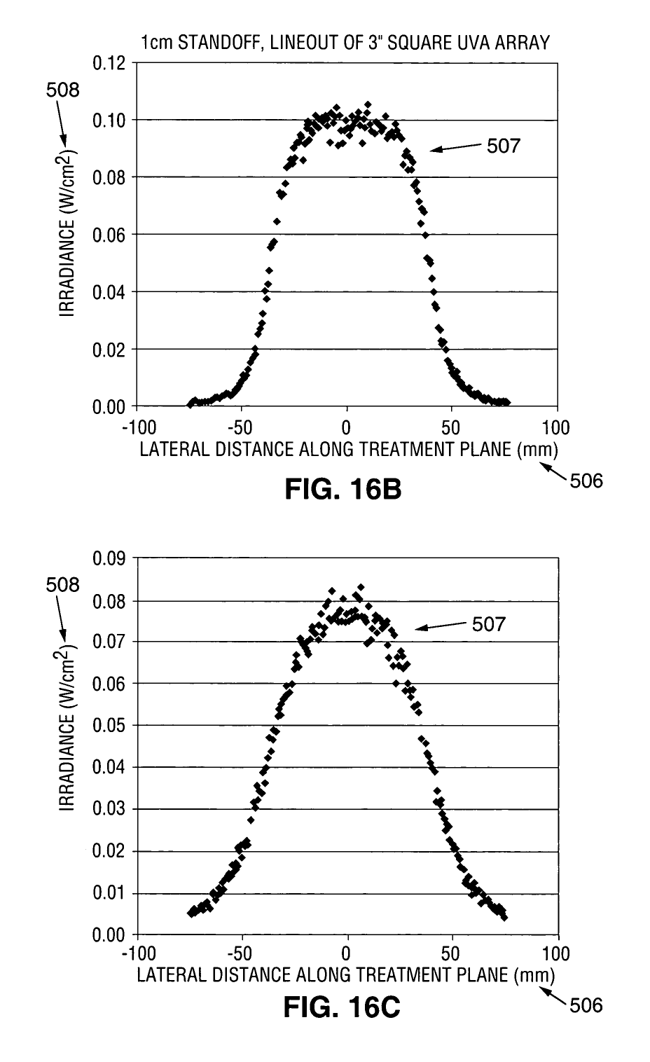

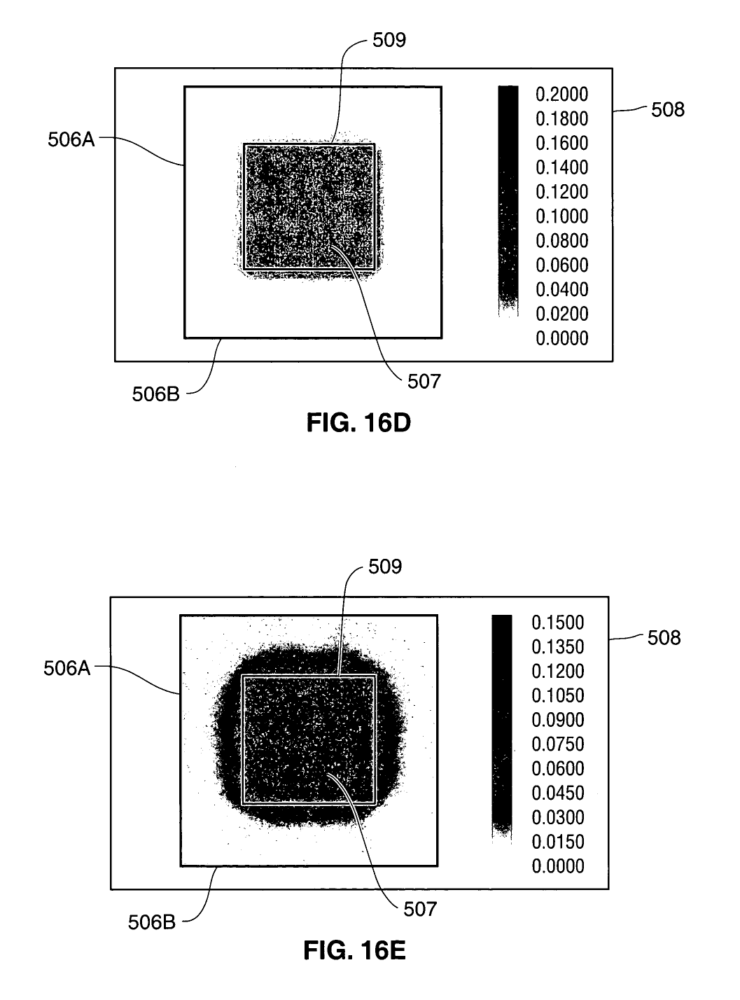

A plot 504 of light energy (optical) power 500 emitted by an LED as a function of current 502 passing though the LED is illustrated in FIG. 16. By way of example, a plot for a Microsemi UPBLED 400 having a central emission light energy wavelength of 400 nm and a continuous current passing through the LED is illustrated in FIG. 16. These LED devices are available from Microsemi, Inc. of Irvine, Calif. In alternate embodiments, any LED having desired output optical power and light wavelengths may be used; for example, a Shark series part number OTL-395A-5-10-66-E multiple emitter LED having a central emission light energy wavelength of 395 nm and a rated output optical power of 250 mW, available from Opto Technology Inc. of Wheeling Ill.; a Lumileds Luxeon Star LXML-MM1C LED having a central emission light energy wavelength of 505 nm and a rated output optical power of 110 mW, available from Lumileds Lighting LLC of San Jose, Calif.; an Osram LV E67C LED having a central emission light energy wavelength of 503 nm and a rated output optical power of 7 mW, available from Osram Opto Semiconductors of San Jose, Calif.; an Osram LT E67C LED having a central light energy emission wavelength of 525 nm and a rated output optical power of 5 mW; a Lumileds Luxeon Star LXML-MM1C LED having a central light energy emission wavelength of 530 nm and a rated output optical power of 43 mW, available from Lumileds Lighting LLC; and a Shark series part number OTL-530A-5-10-66-E multiple emitter LED having a central light energy emission wavelength of 530 nm and a rated output optical power of 72 mW, available from Opto Technology Inc.

In an embodiment, 1000 recently manufactured Microsemi UPBLEDs having a light energy output wavelength of 400 nm are positioned on metal-clad printed circuit board 84 as described above. A potential of 50 V is applied across each ballast circuit. A total of 100 ballast circuits are located on board 84. For each ballast circuit ten LEDs are connected in series with a 47 Ohm ballast resistor as described above. A total of 900 LEDs are located on board 84. A current of 100 mA passes through each ballast circuit. A total current delivered by LED drive circuit 208 is 10 amps. Each LED emits about 6 mW of light energy. The total output power of light energy is about 6 Watts. A dimension 312 across board 84 is about 4 inches (10 cm) and a dimension 312 across array 33 of LED is about 3 inches (7.6 cm). An area of array 33 is about 9 square inches (58 cm²), and the emitted light energy has an average irradiance of about 0.1 Watts/cm² (100 mW/cm²) over the area of the array. For a separation distance 354 of 1 cm, a beam of light energy having a uniform irradiance distribution profile of 100 mW/cm² irradiates a surface 50 of a tissue 52. An acne treatment having a fluence of 100 J/cm² is delivered to a tissue surface in about 1070 seconds (18 minutes).

An irradiance profile distribution at a surface 50 of a tissue 52 is illustrated in FIGS. 16A-16E. An irradiance of treatment beam 42A at a tissue surface is controlled by varying a separation distance 354 between a surface 50 of tissue 52 and board 84. This control is demonstrated by the results of illustrative ray tracing calculations determining an irradiation profile over a tissue surface. Ray tracing calculations to determine an irradiation profile on a tissue surface resulting from light energy emitted from an LED array as described above can be readily calculated by a person of skill. Treatment beam 42A comprises light energy from several spatially overlapping light beams emitted by LED's as described above. A dimension across LED array 33 is three inches (76 mm). An irradiance emitted by the LED's over an area of array 33 is 100 mW/cm². An irradiance distribution profile on a tissue surface for separation distance 354 of 0.5 cm is illustrated in FIG. 16A. A beam irradiance profile distribution 507 having an irradiance 508 is illustrated along position 506 of a surface 50 of a treated tissue 52. A surface 50 of a tissue 52 is irradiated with an irradiance of approximately 100 mW/cm² across a dimension having a length of about 75 mm. A beam irradiance profile distribution 507 for a separation distance 354 of 1 cm is illustrated in FIG. 16B. A surface 50 of a tissue 52 is irradiated with a uniform irradiance of approximately 100 mW/cm² across a dimension having a length of about 60 mm. A beam irradiance profile distribution 507 for a separation distance 354 of 2.5 cm is illustrated in FIG. 16C. A surface 50 of a tissue 52 is irradiated with a uniform irradiance of approximately 70 mW/cm² across a dimension having a length of about 50 mm.

Referring to FIGS. 16D and 16E, an irradiance profile distribution 507 is illustrated as in FIGS. 16A and 16C having separation distances of 0.5 cm and 2.5 cm respectively. A distribution of an irradiance 508 is illustrated along two dimensions 506A and 506B. A square 509 illustrates a size of LED array 33.

In another embodiment LED devices as described above are packed tightly, 1 emitter is placed for each 0.026 Cm² of surface area on board 84. About 40 LED devices are positioned on each square cm of board 84. Each LED emits about 6 mW of optical power. For a 10 cm×10 cm array of LEDs having a surface area of 100 cm², 4000 LEDs are placed in the array. A total optical power output from the array of about 24 Watts is achieved. An irradiance emitted by the array is 240 mW/cm². A light irradiance of 240 mW/cm² is achieved at a tissue surface 50 separated from the array by a separation distance 254 of about 1 cm. Any average light irradiance between 0 and about 240 mW/cm² emitted from a surface area of board 84 can be selected by adjusting drive current 254. For example, emitted light irradiance of at least about 30, 50, 100, 150 and 200 mW/cm² can be selected by adjusting drive current 254. Any average light irradiance between 0 and about 240 mW/cm² at tissue surface 50 can be achieved by selecting drive current 254 and separation distance 354. For example, average tissue treatment power intensities of at least about 30, 50, 100, 150 and 200 mW/cm² can be achieved by selecting drive current 254 and separation distance 354. Any cumulative treatment light fluence can be applied to a tissue surface 50 at any of the above irradiances by selecting a treatment time and an average tissue treatment irradiance. For example, tissue treatments having cumulative fluences of at least about 50 J/cm², 100 J/cm², 150 J/cm² and 200 J/cm² can be achieved. A treatment having a fluence of at least about 100 J/cm² is completed at any average irradiance at a tissue surface 50 between about 30 mW/cm² and 250 mW/cm² by varying separation distance 354 and drive current 254. For example a treatment having an fluence of 100 J/cm² at an average tissue surface irradiance of 200 mW/cm² is completed in 500 seconds (8 minutes).

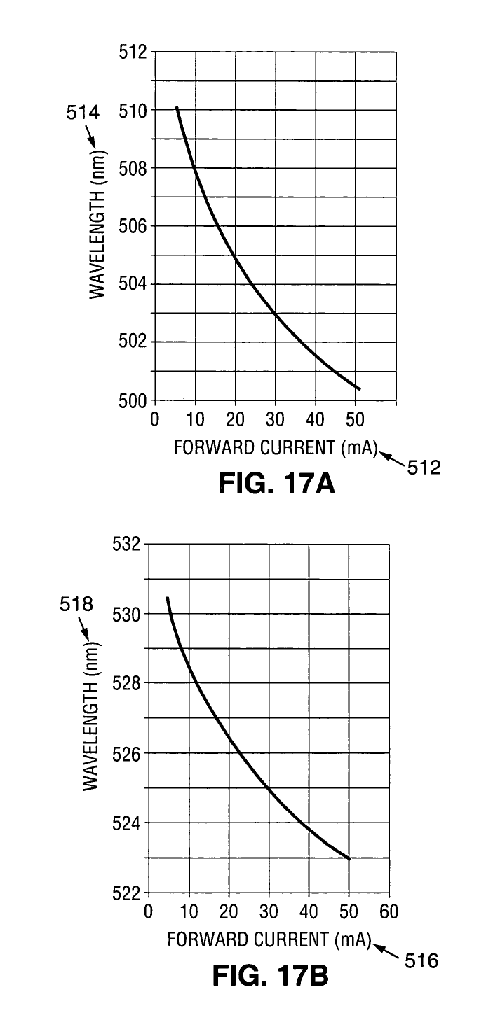

A characteristic of LEDs that can be used to tune a wavelength of a light energy emitted from an LED in preferred embodiments of the invention is illustrated in FIGS. 17A and 17B. Changes in a central wavelength of light energy emitted of as much as 2 nm per 10 mA of current passing through the LED have been reported. A central wavelength of light energy emitted changes with a current passing through an LED. For high power green to ultraviolet LEDs which are generally in the AlGaInN semiconductor material family, several factors contribute to the wavelengths of light energy emitted by an LED. For example, the semiconductor band gap, density of electronic states, levels of impurity donor and acceptor electronic states and thermal/Boltzmann distribution of charge carriers, all contribute to an emission spectrum of an LED. Theoretical descriptions of light energy emission spectral properties have been described and are known. For example, the site “lightemittingdiodes.org” on the world wide web (www) includes a theoretical description of light energies emitted from LEDs.

As a current applied to an LED increases, a central wavelength of light energy emitted changes. As illustrated in FIG. 17A, a green-blue LED available from OSRAM OPTO SEMICONDUCTORS of San Jose, Calif., has a central emission wavelength 514 of 510 nm at an applied forward current 512 of 5 mA. At an increased forward current of about 35 mA, a central wavelength 514 of light energy emitted changes to 502 nm. As similarly illustrated in FIG. 17B, a green LED available from OSRAM OPTO SEMICONDUCTORS has a central emission wavelength 518 of 530 nm at an applied forward current 516 of 5 mA. At an increased forward current of 50 mA, a central emission wavelength 518 changes to about 523 nm. In embodiments of the invention, this characteristic of an LED is advantageously used as will be described in more detail herein below.

Referring to FIG. 4B illustrating fluorescence peaks 74A-74E, a shift in a central emission wavelength is toward a fluorescence peak, for example fluorescence peak 74B. Fluorescence peak 74B is located at an excitation wavelength of about 510 nm. A central emission wavelength 518 as in FIG. 17B is shifted toward a fluorescence peak 74B as a current 516 passing through an LED is increased. For example, central emission wavelength 518 shifts from 530 nm to 523 nm as a current passing through an LED increases from 5 mA to 50 mA. At a current of 50 mA the central emission wavelength 518 is 7 nm closer to the fluorescence excitation peak 74B than at a current of 5 mA and a central emission wavelength of 530 nm.

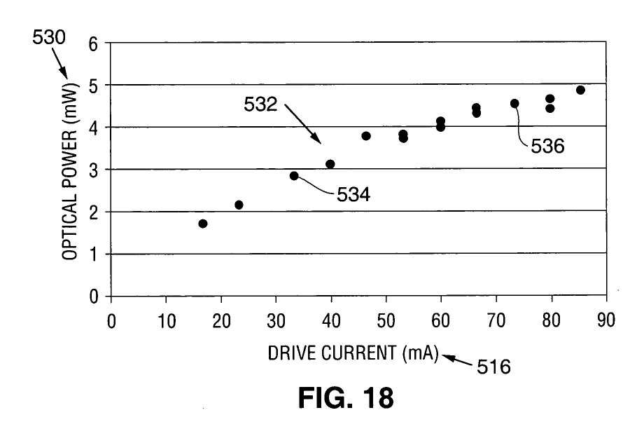

Referring to FIG. 18, a plot 532 of optical power 530 is illustrated for a green LED as in FIG. 17B. Illustrated plot 532 includes measured optical power values 534 and 536 at drive (forward) current 516 passing through an LED. As forward current 516 increases, optical power 530 emitted from an LED increases. A central wavelength of emitted light energy decreases as forward current 516 and light energy power 530 increase. A spectrometer is used to measure a central wavelength and distribution of wavelengths of emitted light energy as forward current 516 is varied. Although any spectrometer can be used for measuring a distribution wavelengths of emitted light energy, a suitable spectrometer is a model USB 2000 available from Ocean Optics, Inc. of Dunedin Fla. This spectrometer uses a calibration white light source model LS-CAL-INT and an integrating sphere model FOIS-1. As illustrated in FIG. 18, drive current 516 is continuous, but may be pulsed in alternate embodiments as described herein below.

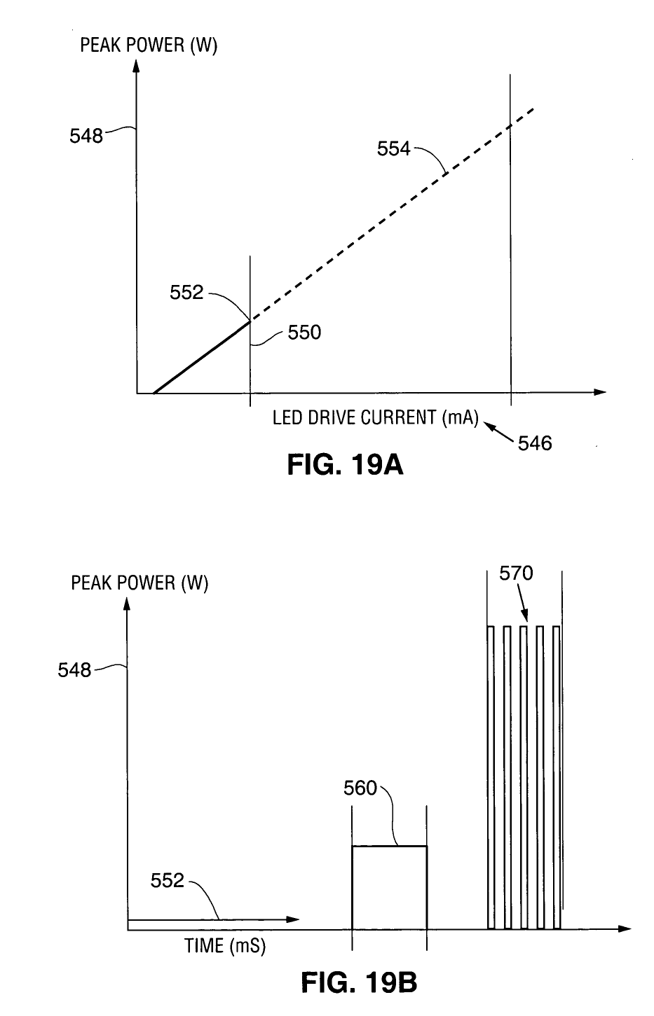

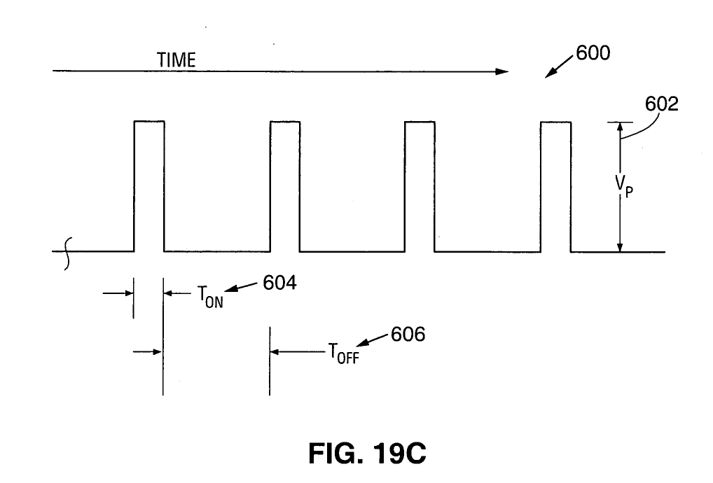

Referring to FIG. 19A a maximum rated current 550 exists for an LED indicates a maximum current at which an LED can be run continuously. A peak power 548 illustrates an instantaneous amount of light energy power emitted by an LED. An LED drive current 546 illustrates an amount of current passing through an LED. A peak output power 548 for an LED running continuously at maximum of drive current 546 is illustrated at 552. In practice it is often desirable to exceed this maximum rated current to increase an amount of light energy power delivered to a tissue and to change a central wavelength and distribution of wavelengths of light energy delivered to a tissue. However, a usable life of an LED is often decreased by exceeding a maximum rated current. Exceeding this maximum rated current is referred to as overdriving an LED. By pulsing a current running through an LED a pulsed peak power 554 of light energy emitted by an LED can be reliably obtained which is greater than a peak output power 552 of an LED running continuously.

Referring to FIG. 19B, an LED is driven continuously at a maximum rated current 552. An LED is driven in a single pulse operational mode 560 with a current above the maximum rated current 550 and a peak output power 548 greater than the peak output power 548 obtained by continuously running the LED at the maximum rated current 550. A multiply pulsed operation 570 produces a peak output power 548 several times greater than the peak output power 548 obtained by continuously running the LED at the maximum rated current 550.

Referring to FIG. 19C, a series of pulses 600 are applied to LEDs during a tissue treatment. Each pulse has a voltage Vp 602 across the ballast circuit 300 as described above. Each pulse lasts a specified duration Ton 604. A specified delay time Toff 606 is a temporal separation between pulses. Voltage Vp 602 is adjusted at control 254 of user interface 22 as described above. A duration 604 and delay 606 of a sequence of pulses 600 is selected with control 22 of user interface 22 as described above.

A selected pulse sequence having a predetermined duration and delay is controlled with processor 202. Processor 202 comprises a computer program adapted to control the duration and delay of a preprogrammed sequence of pulses. By way of example, selectable pulse sequences having specific durations and delays between pulses are illustrated in Table 1 below.

TABLE 1

| Pulse Sequence | Pulse Duration | | Pulse Delay | |
|---|---|---|---|---|
| 1 | CW (continuous) | | CW (i.e. none) | |
| 2 | 100 | ms | 900 | ms |
| 3 | 10 | ms | 90 | ms |
| 4 | 1 | ms | 9 | ms |
| 5 | 100 | μs | 900 | μs |
| 6 | 10 | μs | 90 | μs |
| 7 | 1 | μs | 9 | μs |
| 8 | 1 | ms | 99 | ms |
| 9 | 2 | ms | 8 | ms |
| 10 | 1 | ms | 1 | ms |
| 11 | 10 | ms | 10 | ms |
| 12 | 100 | ms | 100 | ms |
| 13 | 20 | ms | 80 | ms |

As illustrated in Table 1, a duration of an LED pulse ranges from continuous to 1 microsecond (μs) to continuous. Although 13 sequences are illustrated, any pulse duration and delay can be selected. A period of a sequence of pulses encompasses a sum of a pulse time plus a delay time. A duty cycle of a sequence encompasses a pulse time of a sequence divided by a period of a sequence. With the above described technique of shifting an output frequency of an LED, a duty cycle is selected. Although a duty cycle can be 100% (continuous), a range of duty cycles is typically between about 1% and 75%, preferably between about 2% and 50%, more preferably between about 5% and 30% and ideally between about 10% and 20%. In specific embodiments a duty cycle of 15% is used.

In embodiments shifting a central wavelength of emitted light energy, a range of pulse durations is preferably less than 100 ms, more preferably less than 10 ms, even more preferably less than 1 ms, and ideally less than 100 μs. A person of skill can vary a current applied to an LED, change a pulse duration and delay of a sequence of pulses, and measure an output wavelength emitted by an LED as described above to adjust an output wavelength of an LED to more closely match a described wavelength for treating a tissue. Any shift in a central wavelength of emitted light energy between about 1 and about 10 nm can be achieved. For example, shifts in an emitted central wavelength of at least about 3 nm, 5 nm, 7 nm and 9 nm can be achieved.

In an embodiment of the invention, treatment wavelength is selected for a patient based on patient skin color and a desired treatment depth. As illustrated in FIGS. 4A and 4B, optimal treatment wavelengths for the treatment of acne exist at about 510 and 400 nm. For patients with light skin color a treatment at 400 nm is desirable. However, with patients having darker skin, referred to as Type V and Type VI skin, an absorption of light in the ultraviolet near 400 nm can be five times as high as for patients with fair skin. For these skin types, an absorption of light is only about one to three times greater at wavelengths between about 530 and 550 nm. For patients with this darker skin, a treatment light energy having a central wavelength at about 510-520 nm is preferred, especially for treatments to a depth of about 1 mm or deeper as is often desirable in the treatment of acne. A thickness of a skin of a patient can vary with a region of the body. For example, thicker skin is often found on the back and back of the neck of a patient. Structures to be treated within a thick skin are often located at a depth of several millimeters below a surface of the skin, for example structures populated by p. acnes bacteria. Thick skin of patients with both light and dark skin types are preferably treated with light energy having a central wavelength between about 520 and 530 nm. A preferred treatment with a light energy having a central wavelength of about 510 to 520 nm can be performed by adjusting the output wavelength of an LED as described above.

In some embodiments, head 30 may be detachable to permit an exchange of a head having blue LEDs with an output wavelength of about 400 nm for a head having LEDs with a desired blue-green output wavelength of about 515 nm as described above. Alternatively, a module comprising LEDs emitting a blue wavelength of light energy mounted on a first printed circuit board may be made exchangeable with another module comprising LEDs emitting a blue-green wavelength of light energy mounted on a second printed circuit board.

Although the above provides a complete and accurate description of specific preferred embodiments of the invention, modifications and changes can be made that are still within the scope of the invention. For example, although specific reference has been made to treating acne with specific wavelengths of light, any exposed tissue may be treated with light having a wavelength between about 380 and 800 nm in accord with the above described invention. Therefore, the scope of the invention is limited solely by the following claims.

1-62. (canceled)

63. A system for treating the tissue of a patient with light, said system comprising:
an array of light emitting diodes;
a circuit board having a patterned, electrically conductive layer to which said diodes are mounted in the form of a densely packed array, said circuit board further including a dielectric layer supporting said patterned conductive layer, said circuit board further including a heat conducting, metal layer supporting said dielectric layer;
an actively cooled heat sink, thermally connected to the metal layer of said circuit board for cooling the diodes;
a housing in which said diodes, said circuit board and said heat sink are mounted; and
a drive circuit for powering the diodes in a manner so that the diodes emit light for treating the tissue of the patient.

64. A system as recited in claim 63, wherein the metal layer of the circuit board is formed from aluminum.

65. A system as recited in claim 63, wherein the heat sink is a fluid cooled block having fluid channels formed therein.

66. A system as recited in claim 65, wherein said block is formed from aluminum.

67. A system as recited in claim 63, further including a transmissive optical element mounted to said housing and separating the diodes from the tissue.

68. A system as recited in claim 67, wherein the optical element is a lens.

69. A system as recited in claim 63, wherein a spacer is connected to the housing to define a predetermined separation from the diodes and the tissue.

70. A system as recited in claim 63, wherein a drive circuit is connected to a processor to control the light output reaching the tissue.

71. A system for treating the tissue of a patient with light, said system comprising:
an array of light emitting diodes;
a circuit board having a patterned, electrically conductive layer to which said diodes are mounted in the form of a densely packed array, said circuit board further including a dielectric layer supporting said patterned conductive layer, said circuit board further including a heat conducting, metal layer supporting said dielectric layer;
a fluid cooled block, thermally connected to the metal layer of said circuit board for cooling the diodes;
a housing in which said diodes, said circuit board and said heat sink are mounted;
a transmissive optical element mounted to said housing and separating the diodes from the tissue;
a drive circuit for powering the diodes in a manner so that the diodes emit light for treating the tissue of the patient; and
a processor connected to the drive circuit for controlling the drive circuit.

72. A system as recited in claim 71, wherein the metal layer of the circuit board is formed from aluminum.

73. A system as recited in claim 71, wherein said block is formed from aluminum.

74. A system as recited in claim 71, wherein the optical element is a lens.

75. A system as recited in claim 71, wherein a spacer is connected to the housing to define a predetermined separation from the diodes and the tissue.
